(19) United States
(12) Patent Application Publication
(10) Pub. No.: US 2003/0045790 A1
(43) Pub. Date: Mar. 6, 2003

(54) System and method for three dimensional display of body lumens

(76) Inventors: Shlomo Lewkowicz, Kiryat Tivon (IL); Arkady Glukhovsky, Nesher (IL); Daphna Levy, Carmiel (IL); Gavriel Meron, Petach Tikva (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 10/234,141

(22) Filed: Sep. 5, 2002

Related U.S. Application Data
(60) Provisional application No. 60/316,950, filed on Sep. 5, 2001.

(51) Int. Cl. A61B 5/05

(52) U.S. Cl. 600/407

(57) ABSTRACT

A method and system for positioning an in vivo device and for obtaining a three dimensional display of a body lumen include obtaining a plurality of in vivo images; generating position information corresponding to each in vivo image; and combining the plurality of in vivo images into a single image according to the position information.

### PRIOR PROVISIONAL APPLICATION

The present application claims benefit from prior provisional application No. 60/316,950, entitled “SYSTEM AND METHOD FOR THREE DIMENSIONAL DISPLAY OF BODY LUMENS” and filed on Sep. 5, 2001.

### FIELD OF THE INVENTION

The present invention relates to the field of endo-luminal sensing. More specifically, the present invention relates to a system and method for real-time position and orientation monitoring and to image generation of body lumens.

### BACKGROUND OF THE INVENTION

Endo-luminal imaging greatly enhances a practitioner's ability to safely and easily view internal body features and occurrences with minimal intrusion.

Body lumens, especially voluminous cavities, may be most effectively viewed when an image (preferably a three dimensional image) of the entire cavity may be displayed and internal features and occurrences may be easily located within a lumen according to a known position of an in vivo imaging device.

### SUMMARY OF THE INVENTION

A system according to one embodiment of the invention, includes an in vivo sensing device, such as an imaging device, which includes at least one position monitor for generating position information, preferably three dimensional position information, of the autonomous in vivo device at any given time; a receiving unit for receiving position information from the position monitor and optionally for receiving in vivo data from the sensing device; and a processing unit for computing the position and/or orientation of the in vivo device at any given time.

According to another embodiment the invention provides a system and method for obtaining a three dimensional display of in vivo images. Further, according one embodiment, a panoramic view of a body lumen can be displayed using the system and method according to an embodiment of the invention, typically by employing “real time mapping” and image mosaic constructing techniques.

### BRIEF DESCRIPTION OF THE DRAWINGS

The present invention will be understood and appreciated more fully from the following detailed description taken in conjunction with the appended drawings in which:

FIG. 1 is a schematic illustration of the system according to an embodiment of the invention;

FIG. 2 is a schematic illustration of an imaging device according to an embodiment of the invention; and

FIG. 3 is a flow diagram illustrating the steps of a method according to an embodiment of the invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

According to one embodiment, the present invention combines positioning methods and image processing methods in an in vivo imaging system for obtaining a three dimensional in vivo image, especially, for example, for obtaining images of relatively voluminous body lumens, such as the stomach or large intestine.

A system according to an embodiment of the invention, includes an imaging device for obtaining a plurality of endo-luminal images, the imaging device, according to one embodiment having at least one position monitor for generating position information, e.g., three dimensional position information, corresponding to each endo-luminal image; a receiving unit for receiving position information from the position monitor and optionally for receiving image data from the imaging device; and a processing unit for computing the position and/or orientation of the imaging device corresponding to each endo-luminal image and for combining the plurality of endo-luminal images into a single image, optionally a mosaic image, according to the position and/or orientation of the imaging device when each endo-luminal image was obtained. The position information and/or the image data can be transmitted, for example, wirelessly or through a wired connection to the receiving unit.

The single combined image is typically a three dimensional image. Thus, a plurality of images, each of a different portion of a body lumen may be combined to a single, preferably three dimensional, image of for example, an entire body lumen.

According to one embodiment the imaging device may include a unit for receiving commands and for moving or positioning the imaging device in accordance with the received commands. According to one embodiment the commands may be sent by an external operator. According to another embodiment the commands may be automatically generated by the processing unit in accordance with the position information which is received and processed by the processing unit.

According to certain embodiments of the invention a system includes an autonomous in vivo device, optionally an in vivo sensing device, such as an image sensor, a pH meter, a pressure detector, a thermometer etc., which includes at least one position monitor for generating position information, preferably three dimensional position information, of the autonomous in vivo device at any given time; a receiving unit for receiving position information from the position monitor and optionally for receiving in vivo data from the sensing device; and a processing unit for computing the position and/or orientation of the in vivo device at any given time. The position information and/or the in vivo data can be transmitted, for example, wirelessly or through a wired connection to the receiving unit. The autonomous in vivo device may be a swallowable capsule capable of sensing the GI environment and/or capable of performing in vivo procedures.

A method according to an embodiment of the invention includes the steps of obtaining a plurality of endo-luminal images from an imaging device; generating position information, preferably three dimensional position information, of the imaging device corresponding to each endo-luminal image; receiving the position information; computing the position and/or orientation of the imaging device corresponding to each endo-luminal image; and combining the plurality of endo-luminal images into a single image according to the position and/or orientation of the imaging device when each endo-luminal image was obtained. The method may include a further step of controlling movement or the position of the imaging device, preferably according to the obtained image and/or position information. The method, according to another embodiment of the invention, includes the steps of inserting into a body lumen an autonomous in vivo device; generating position information, preferably three dimensional position information, of the in vivo device at any given time; receiving the position information; computing the position and/or orientation of the in vivo device at any given time. The method may further include a step of controlling movement or the position of the in vivo device, preferably according to the position information.

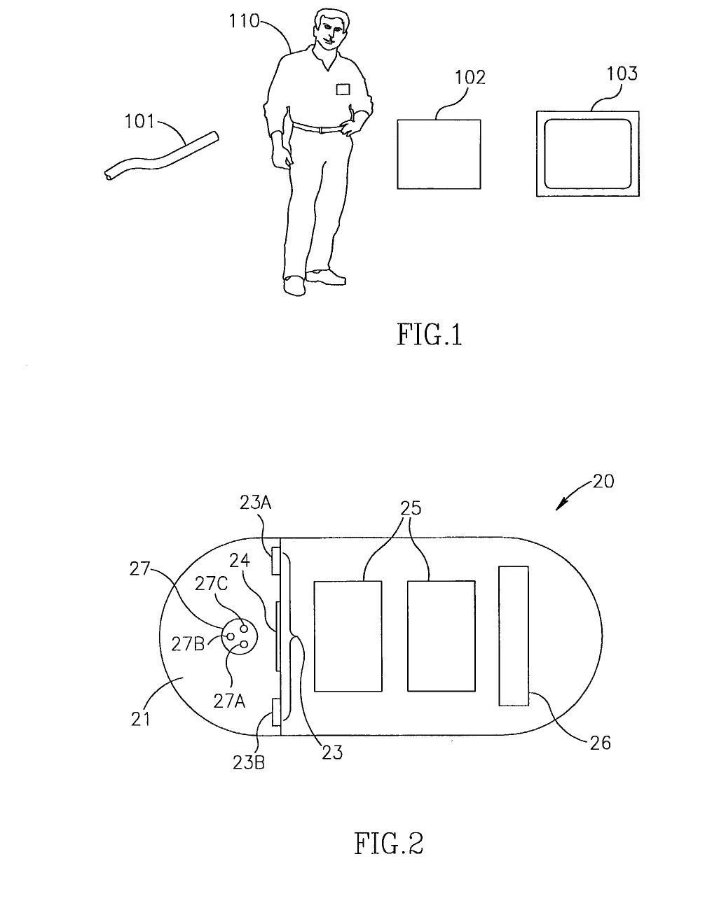

Reference is now made to FIG. 1 in which a system according to an embodiment of the invention includes, for example, an in vivo imaging device 101, a receiving and processing unit 102 and a display 103. The imaging device 101, in the embodiment illustrated in FIG. 1, is typically part of an endoscope or catheter that is inserted into a patient 110 for imaging and possibly otherwise sensing body lumens, such as the GI tract, blood vessels, the reproductive tract or any other suitable body lumens. The imaging device 101 typically includes an image sensor (not shown) such as a CCD or a CMOS image sensor, an illumination source for illuminating an in vivo site (not shown) and a transmitter (not shown) for transmitting image data to the receiving and processing unit 102. Data may be transmitted wirelessly or through a wired connection. The imaging device 101 may further include in vivo sensors, such as a pH meter, temperature sensors, pressure sensors and so on, for sensing the endo-luminal environment. Sensed endo-luminal conditions may be transmitted (wirelessly or not) to the receiving and processing unit 102. Examples of in vivo sensing systems that can be utilized in embodiments of the present invention are described in U.S. Pat. No. 5,604,531 to Iddan and in International Application Publication No. WO0165995 published Sep. 13, 2001, to Glukhovsky, both of which are assigned to the common assignee of the present invention and which are incorporated by reference. The systems described above may be battery operated and wireless or may be connected to a power supply and/or light source external to the patient's 110 body.

According to one embodiment the imaging device 101 also includes a position monitor (not shown) for indicating the position and orientation of the imaging device 101 in the body lumen. Typically, the position monitor included in the imaging device 101 includes at least three receivers or transceivers and a sensing device for distinguishing between signals received by the different transceivers.

The transceivers or other position monitoring devices that are included in the imaging device 101 are typically part of a position monitoring system, which also includes an external reference frame. The external reference frame typically includes transmitters, such as electromagnetic or acoustic transmitters, at known positions in the reference frame, for transmitting signals that are received by the transceivers in the position monitor included the imaging device 101. The external reference frame may be part of the receiving and processing unit 102, which is typically located in proximity to patient 110. The receiving and processing unit 102 further includes, according to an embodiment of the invention, a receiving system for receiving image data from the image sensor and/or other in vivo sensors in imaging device 101 and, optionally, receivers for receiving signals from the transceivers. Signals from the transceivers may be transmitted to receiving and processing unit 102 for calculating position information or alternatively, the position monitor (in imaging device 101) may include processing devices for calculating position information from the signals received by the transceivers. The position information typically includes six degrees of freedom, providing information about the imaging device's location and moment. Any suitable position monitoring systems known in the art can be utilized in embodiments of the present invention. Examples of position monitoring systems that can be easily adjusted for use with embodiments of the present invention are described in U.S. Pat. No. 5,697,377 to Wittkampf, U.S. Pat. No. 5,515,853 to Smith and U.S. Pat. No. 6,188,355 to Gilboa. These U.S. patents are hereby incorporated by reference. Examples of calculation methods applicable in embodiments of the present invention are described in WO 01/06917 to Gilboa and WO 00/10456 to Blecher et al. Both publications are hereby incorporated by reference. It will be appreciated that the calculations are carried out on suitable computational or processing devices.

According to one embodiment receiving and processing unit 102 also includes image processing modules for combining a plurality of typically non consecutive endo-luminal images obtained and transmitted by the imaging device 101 into a single three dimensional image of substantially the entire body lumen. Any suitable image processing procedures may be used in embodiments of the present invention, such as computing local motion estimates between pairs of overlapping images, registration, “gap closing”, identification of overlapping portions of images, warping input images and aligning sets of overlapping images to construct a mosaic image, as known in the art. Images may be divided into patches and some of the computations of optimization processes can be done on a per-patch basis or the computation and optimization processes can be performed for each and every pixel. Known image mosaic constructing procedures may be adjusted for use in embodiments of the present invention. The receiving and processing unit 102 may further include a transmitting mode for transmitting commands to the position monitoring device for controlling the position of the imaging device 101, as known in the art.

Controlling an in vivo device may be performed as known in the art. For example, portions of the imaging device may be made of a shape memory material whereas heating portions of the device, for example by passing a current through a conducting element in the vicinity of these portions, may controllably move the device. Alternatively, the device may include a magnet whereas applying an external magnetic field may control the device as known in the art.

According to one embodiment the combined image and/or any other information, such as positional information or information relating to endo-luminal environmental conditions are displayed on display 103, which may be a part of the receiving and processing unit 102 (such as a screen of a computer or a video monitor) or a separate LCD or any other suitable display.

An imaging device in accordance with another embodiment of the invention is schematically illustrated in FIG. 2. The imaging device 20 is, for example, an ingestible capsule, such as the devices described in the above mentioned U.S. Pat. No. 5,604,531 and International Application Publication No. WO0165995. The imaging device 20 includes an illumination unit 23, typically comprising a plurality of illumination sources such as white LEDs 23A and 23B, an optical sensor 24, a transmitter 26 for transmitting image signals of the image sensor 24, a position monitor 27 and a power source 25, such as a silver oxide battery, that provides power to the entirety of the electrical elements of the imaging device 20. The device 20 may include other configurations and other components.

The imaging device 20 is typically capsule shaped, can be easily swallowed and may passively pass through the entire GI tract. While passing through tube like portions of the GI tract, such as the small intestine, the imaging device 20 may be pushed along by natural peristalsis and may be restricted by the tube walls to a fixed orientation. As the imaging device 20 passes through the small intestine it may periodically image the tube wall. However, when the imaging device 20 reaches cavities such as the stomach or the large intestine it is no longer restricted by the lumen walls and it may rotate and tumble through the lumen periodically imaging different, not necessarily consecutive portions of the lumen wall. The orientation of the imaging device 20, and particularly of the image sensor 24, each time the lumen is imaged, can be determined by the position monitor 27. According to an embodiment of the invention the position monitor 27 includes three electrodes or coils or transponders 27 A-C that receive electromagnetic signals transmitted from an external source. The external source may typically include three electromagnetic transmitters at a fixed position in an external reference frame, that transmit three distinguishable electromagnetic radiations (such as at different frequencies). The electrodes 27 A-C receive signals corresponding to the electromagnetic radiations at a plurality of times, each of the signals including components of at least one of the three radiations. The electrodes 27 A-C form functions that include the components of the signal received by the each electrode from the three transmitters. The position and the orientation of the imaging device 20 is inferred from the functions, as further elaborated in the above mentioned U.S. Pat. No. 6,188,355.

Other position monitors may be used with embodiments of the present invention, such as monitors that include ultrasound transceivers or monitors that include, for example, three magnetic coils that receive and transmit positional signals relative to an external constant magnetic field. For example, magnetic marker monitoring techniques may be used as described in a paper published by Weitschies et al. (Weitschies et al (2001) European Journal of Pharmaceutical Sciences 13, 411-416), which is hereby incorporated by reference.

According to one embodiment position information received from position monitor 27 is used in processing the images obtained by the imaging device 20 in the stomach or large intestine, as described above. The imaging device 20 may further include a controller for synchronizing the operation of the position monitor 27 with the arrival of the imaging device 20 in the stomach or large intestine. Further, a controller may apply suitable algorithms for assigning a specific image to a specific position and/or orientation of the imaging device 20.

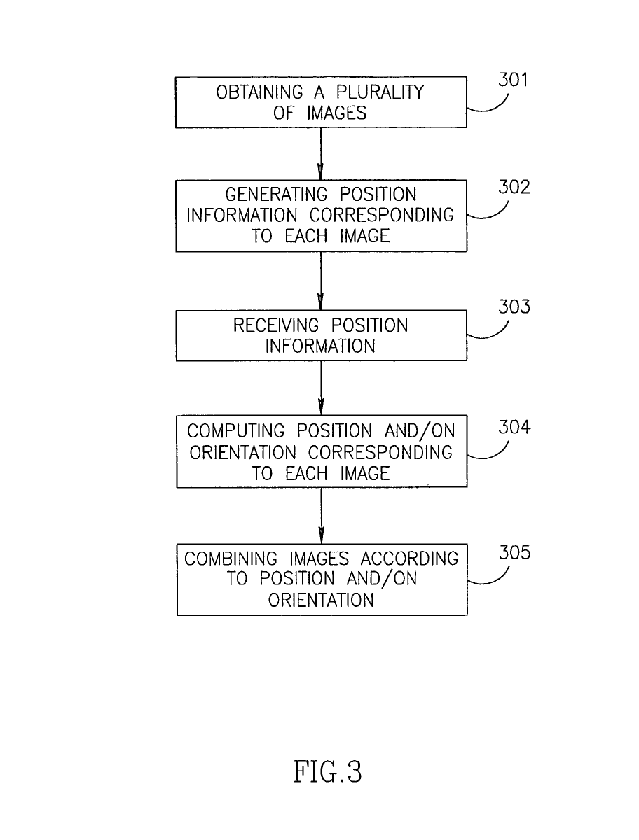

A method for obtaining a three dimensional display of a body lumen, in accordance with an embodiment of the invention, is schematically presented in FIG. 3. The method according to one embodiment includes the steps of obtaining a plurality of endo-luminal images from an imaging device (301); generating position information of the imaging device corresponding to each endo-luminal image (302); receiving the position information (303); computing the position and/or orientation of the imaging device corresponding to each endo-luminal image (304); and combining the plurality of endo-luminal images into a single image according to the position and/or orientation of the imaging device when each endo-luminal image was obtained (305).

Other steps or series of steps may be used.

As described above, endo-luminal images are obtained by an imaging device that sends information to a position monitor. The position monitor generates position information, which is transmitted to a receiving and processing unit. The position and/or orientation of the imaging device is computed, for example by utilizing image mosaic techniques as known in the art and as described above and the computed position is used to correctly assemble and combine the plurality of images into a single image of the body lumen.

According to another embodiment the method may include a further step of controlling movement or the position of the imaging device (as described above), preferably according to the obtained image and/or position information.

It will be appreciated by persons skilled in the art that the present invention is not limited by what has been particularly shown and described herein above. Rather the scope of the invention is defined by the claims which follow.

1. An in vivo imaging system comprising:
at least one imaging device;
at least one position monitor;
a receiving unit configured for receiving position information from the position monitor; and
a processing unit for computing the position and orientation of the imaging device.

2. The system according to claim 1 wherein the receiving unit is capable of receiving image data from the imaging device.

3. The system according to claim 1 wherein the device obtains a plurality of in vivo images.

4. The system according to claim 3 wherein the receiving unit is capable of receiving position information that corresponds to each of the plurality of in vivo images obtained by the device.

5. The system according to claim 3 wherein the processing unit is capable of combining the plurality of in vivo images into a single image.

6. The system according to claim 5 wherein the single image is a mosaic image.

7. The system according to claim 3 wherein the processing unit is capable of combining the plurality of in vivo images in accordance with the position and orientation of the imaging device that corresponds to each of the plurality of images.

8. The system according to claim 1 wherein the position monitor comprises an external reference frame, said reference frame comprising transmitters at known positions, said transmitters configured for transmitting signals to the position monitor.

9. The system according to claim 1 wherein the position monitor comprises three elements configured for receiving electromagnetic signals transmitted from an external source.

10. The system according to claim 9 wherein the external source comprises a plurality of transmitters, said transmitter being at a fixed position in an external reference frame.

11. The system according to claim 1 wherein the system is wireless.

12. An autonomous in vivo imaging device comprising:
at least one image sensor;
at least one illumination source;
at least one transmitter configured for transmitting image signals to an external receiving unit; and
at least one position monitor configured for transmitting position data.

13. An autonomous in vivo sensing system comprising:
a sensing device;
a position monitor;
a receiving unit; and
a processing unit for computing the position and orientation of the sensing device.

14. The system according to claim 13 further comprising an external reference frame, said reference frame comprising transmitters at known positions configured for transmitting signals to the position monitor.

15. The system according to claim 13 wherein the sensing device is selected from a group consisting of a pH meter, a temperature sensing device and a pressure sensing device.

16. The system according to claim 13 wherein the sensing device is an image sensor.

17. A method for obtaining a three dimensional display of a body lumen, said method comprising the steps of:
obtaining a plurality of in vivo images;
generating position information corresponding to each in vivo image; and
combining the plurality of in vivo images into a single image according to the position information.

18. The method according to claim 17 wherein the step of combing the plurality of images is performed by computing local motion estimates between pairs of overlapping images or registration or gap closing or identification of overlapping portions of images or warping input images and aligning sets of overlapping images to construct a mosaic image.

19. The method according to claim 17 further comprising the step of transmitting the position information.

20. The method according to claim 17 wherein the step of obtaining a plurality of in vivo images is performed by an in vivo imaging device.

21. The method according to claim 19 further comprising the step of controlling the position of the imaging device.

22. The method according to claim 20 wherein the imaging device position is controlled in accordance with position information.

23. A method for positioning an in vivo device, said method comprising the steps of:
generating position information of the in vivo device at any given time, said position information generated by a position monitor comprising three elements configured for receiving electromagnetic signals transmitted from an external source; and
computing the position and orientation of the in vivo device at any given time.

24. The method according to claim 22 further comprising the step of controlling position of the in vivo device.

25. An autonomous in vivo sensing system comprising:
a sensing device;
a position monitor;
a receiving unit means for receiving information from the sensing device; and
a processing unit means for computing the position and orientation of the sensing device.

26. An autonomous in vivo imaging device comprising:
an image sensor;
an illumination source;
a transmitter means for transmitting image signals to an external receiving unit; and
a position monitor means for transmitting position data.

27. An in vivo imaging system comprising:
at least one imaging device;
at least one position monitor;
a receiving unit means for receiving position information from the position monitor; and
a processing unit means for computing the position and orientation of the imaging device.

28. An in vivo imaging system comprising:
at least one imaging device;
at least one position monitor;
a receiving unit configured for receiving position information from the position monitor; and
a processing unit capable of computing the position and orientation of the imaging device and capable of combining the plurality of in vivo images into a single image.

29. An in vivo imaging system comprising:
at least one imaging device;
at least one position monitor;
a receiving unit configured for receiving position information from the position monitor; and
a processing unit for computing the position and orientation of the imaging device;
wherein the processing unit is capable of combining a plurality of in vivo images in accordance with the position and orientation of the imaging device that corresponds to each of the plurality of images.

30. A method for obtaining a three dimensional display of a body lumen, said method comprising the steps of:
obtaining a plurality of in vivo images;
generating position information corresponding to each in vivo image; and
combining the plurality of in vivo images into a single image according to the position information;
wherein the step of combing the plurality of images is performed by computing local motion estimates between pairs of overlapping images or registration or gap closing or identification of overlapping portions of images or warping input images and aligning sets of overlapping images to construct a mosaic image.
